(19) United States
(12) Patent Application Publication
(10) Pub. No.: US 2013/0269693 A1
(43) Pub. Date: Oct. 17, 2013

(54) Method And Apparatus For Controlling The Delivery Of Humidified Air

(71) Applicants: Joel David Neatrour, Johnstown, PA (US); Michael Knepper, Friedens, PA (US)

(72) Inventors: Joel David Neatrour, Johnstown, PA (US); Michael Knepper, Friedens, PA (US)

(73) Assignee: DEVILBISS HEALTHCARE LLC, Somerset, PA (US)

(21) Appl. No.: 13/449,005

(22) Filed: Apr. 17, 2012

(51) Int. Cl. A61M 16/16

(52) U.S. Cl. 128/203.12

(57) ABSTRACT

An improved humidification method and apparatus for use with a CPAP type machine in which humidified air is delivered to the patient only during the inhalation phase and non-humidified air is delivered to the patient during the exhalation phase. The invention consists of a valve apparatus to switch from humidified air to non-humidified air and a method of determining when to switch the state of the valve.

### FIELD OF THE INVENTION

This invention is related to the field of breathing gas delivery machines, such as continuous positive airway pressure (CPAP) or bi-level positive airway pressure (Bi-PAP) machines of the type typically used to treat patients suffering from breathing disorders, such as hypopnea or apnea, and, in particular, is related to the humidification apparatus and methods for such devices.

### BACKGROUND OF THE INVENTION

Continuous Positive Airways Pressure (CPAP) machines are well known in the art for use in the treatment of a number of respiratory conditions, such as sleep apnea and hypopnea, by supplying a continuous positive pressure to a patient's airway while the patient sleeps.

It is well known in the art that such machines be equipped with a humidification apparatus to humidify the air being delivered to the patient. This tends to increase the comfort of the patient and eliminates the “dry mouth” condition experienced by many users of the machines.

A typical humidification apparatus consists of a simple humidification chamber containing a reservoir into which water is introduced by the user. The water is heated by an electro-resistive heating element and the air flow being delivered to the patient passes through the humidification chamber containing the heated water, thereby warming and humidifying the air. The device may be equipped with multiple settings settable by the user to vary the level of humidification by varying the temperature to which the water is heated.

There are several problems existing with the current humidification devices for the CPAP machines. First users may experience a condition known as “rain-out” in which heated humidified air exits the humidification chamber and condenses as it cools in the lower temperature hose before reaching the patient. The condensed water tends to accumulate in the hose and may even block certain portions of the hose, forcing the user to breath are through the accumulated water, creating a “gurgling” sound and interfering with pressure delivery to the patient. This problem is sometime solved in the prior art by providing a heated wire along the length of the tube to prevent the air from cooling as it flows through the tube. However, this solution is undesirable in that it tends to increase the overall power consumption of the machine and cost of the air supply tube which is usually disposable and replaced at set intervals.

In addition to the rain-out problem, the heating of the water also increases the overall power consumption of the machine. Therefore it would be desirable to provide an improved humidification apparatus that address both the rain-out problem without increasing power consumption.

### SUMMARY OF THE INVENTION

The present invention includes a method operating a CPAP machine to improve the humidification of the air being breathed by the user, as well as the accompanying improved humidification apparatus.

As is well known to one skilled in the art, the breathing cycle of a patient is composed of an inhalation phase and a exhalation phase. In prior art devices, the patient airflow is permitted to pass through the humidification chamber during the entire breathing cycle, both during the inhalation phase and the exhalation phase. In the preferred embodiment of the present application, the patient air flow is allowed to pass through the humidification chamber only during the inhalation phase of the breathing cycle. During the exhalation phase of the breathing cycle, the air flow is diverted to an alternate path that does not pass through the humidification chamber.

This modified method of handling the airflow addresses both problems noted above. With respect to the rain-out problem, the cooler, dryer air passing through the hose during the exhalation phase of the breathing cycle will tend to dry out any condensing humidity in the hose and secondly, the passage of less air through the humidification chamber will cause the electro-resistive heater to use less energy to keep the water in the reservoir heated to the desired temperature, thereby not only reducing the likelihood of rainout but actually reducing power consumption at the same time.

In addition, the method provides the added benefit of requiring the user to fill up the water reservoir in the humidification chamber less often. In empirical studies the method of the present invention can reduce both the water consumption and the power consumption of the device by over 50%.

### BRIEF DESCRIPTION OF THE DRAWINGS

FIG. 1 is a block diagram of a CPAP machine containing the improved humidifying apparatus of the present invention.

FIG. 2 is a flow chart of the method of the present invention.

FIG. 3 is a cross-sectional perspective view of a valve which may be used to implement the present invention.

FIG. 4 is a partially transparent view of the valve in FIG. 3 in the first position.

FIG. 5 is a partially transparent view of the valve in FIG. 3 in the second position.

FIGS. 6a and 6b show a perspective and end view respectively of the valve shuttle portion of the valve in FIG. 3.

FIG. 7 is a view of the outside of the valve of FIG. 3.

### DETAILED DESCRIPTION OF THE INVENTION

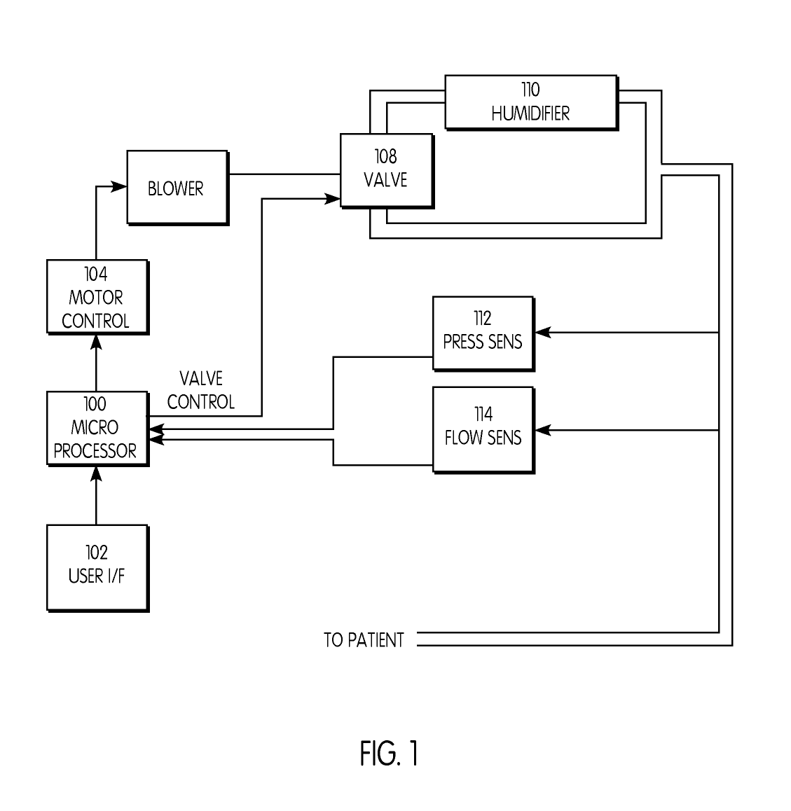

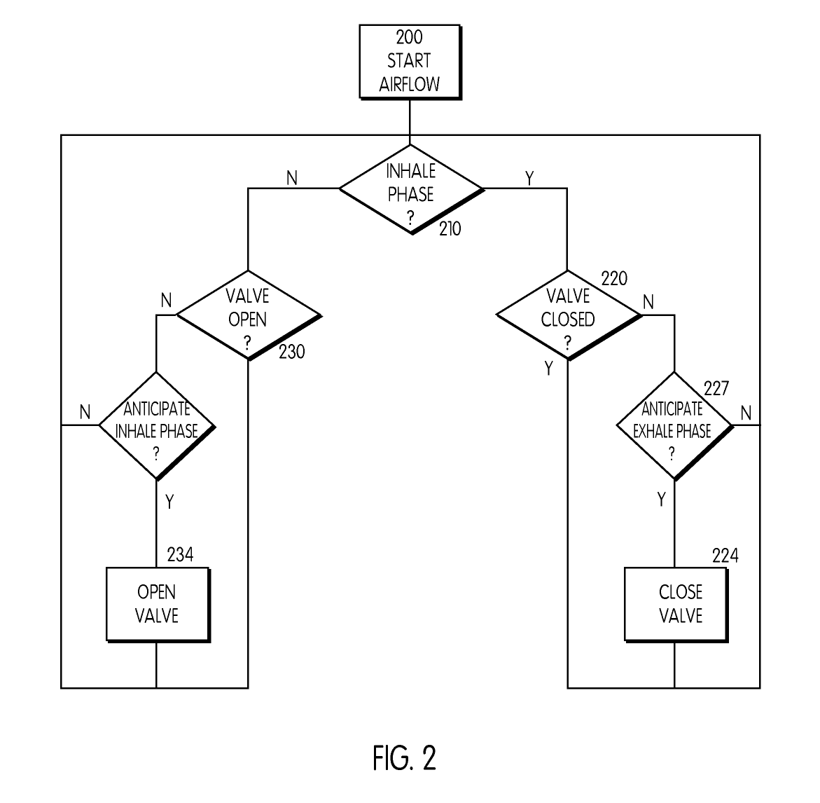

FIG. 1 shows a block diagram of a CPAP machine containing the improved humidifying apparatus of the present invention. The machine is controlled by microprocessor 100. Microprocessor 100 controls blower 106 through motor control 104 to control both the pressure and the airflow rate delivered to the patient. Pressure sensor in 112 and flow sensor 114 are utilized by microprocessor 100 to determine when the inhalation and exhalation phases of the breathing cycle occur. These sensors may be located anywhere between blower 106 and the patient. The user interface 102 is coupled to microprocessor 100 and allows use to set certain parameters of the machine, including the humidification level.

In a preferred embodiment of the invention, the microprocessor 100 also controls solenoid-enable valve 108 which can be in one of two states. In the first state, valve 108 allows airflow to pass from blower 106 through humidifier 110. In the second state, valve 108 diverts the airflow away from humidifier 110 and sends it directly to the patient.

Many CPAP-type machines contain algorithms to detect the beginning and end of the inhalation and exhalation phases of the breathing cycle of the user. In particular, in Bi-PAP devices this detection is necessary because different pressures are applied during the inhalation and exhalation phases. The actual method of detecting the transition between the inhalation and exhalation phases is not part of this invention. However, the presence of such an algorithm and its implementation in the device is necessary to take advantage of the method and apparatus of the present invention.

In one embodiment of the invention, valve 108 may switch from one state to the other when the phase transition is detected. However, in a preferred embodiment, it is desirable to open or close the valve in anticipation of the phase transition such that humidified air reaches the patient at the approximate start of the inhalation phase and non-humidified air reaches the patient at the approximate start of the exhalation phase. Switching the state of valve 108 at the transition detection instead of in anticipation of the transition will cause a short overlap at the beginning of each phase, during which, for example, humidified air is being delivered to the patient when non-humidified air is desired, or visa versa.

Thus, the opening and closing of valve 108, which allows the airflow to either pass through humidification chamber 110 or be diverted therefrom, need to occur in anticipation of the transitions instead of when the transition between the phases is actually detected. Various factors, such as the length of the tube between the machine and the user and the amount of leakage being experienced by the user will effect the time it takes for humidified air to travel from humidification chamber 110 to the patient. Therefore, the actual time period in anticipation of the transition will be dependent upon these and other factors.

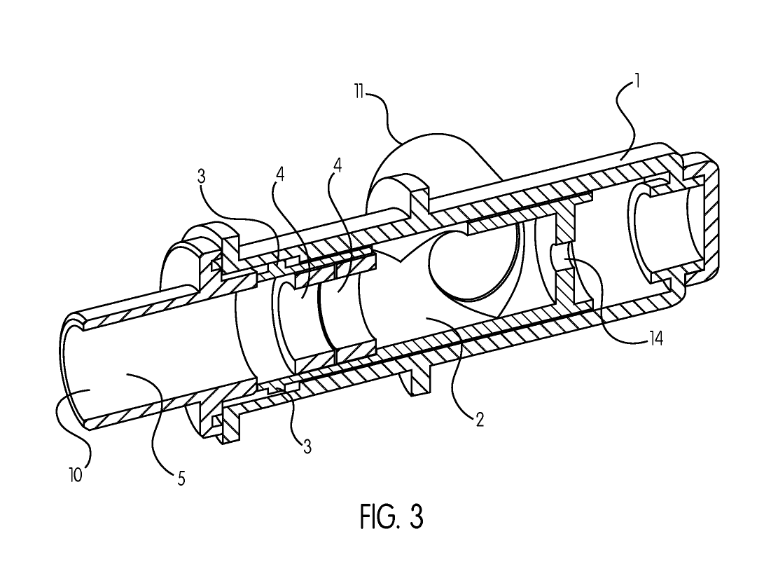

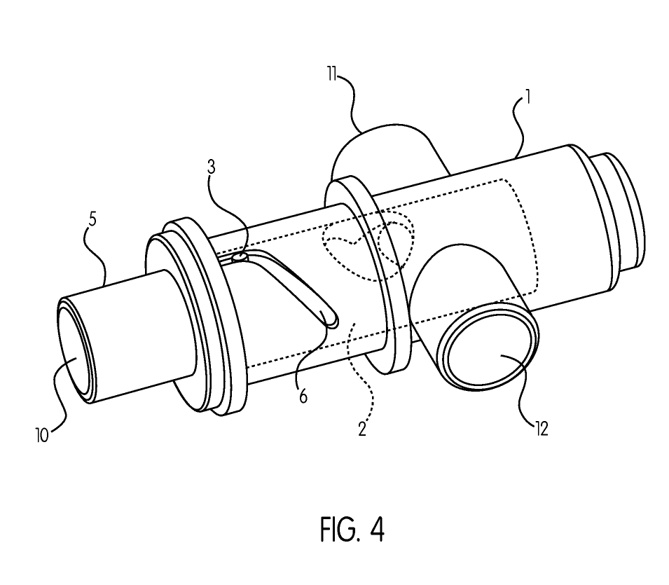

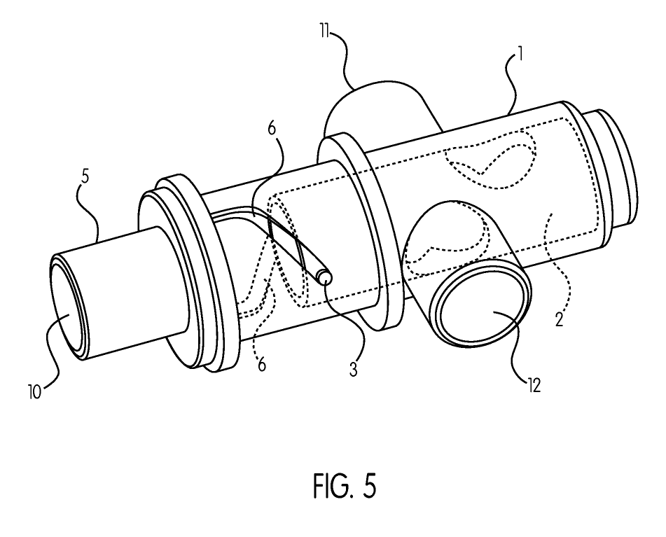

A preferred embodiment of the valve is shown in FIGS. 3 through 7, however, as would be realized by one of skill in the art many of the implementations of the valve are possible. The valve consists of valve body 1, of tubular construction having inlet port 10 defined at one end thereof and outlet ports 11 and 12 defined on opposite sides thereof as best shown in FIGS. 4, 5, and 7. The valve is designed such that air flows in inlet port 10 and out either outlet port 11, which may, for example, be connected to a bypass pathway that bypasses humidification chamber 110 or through outlet 12 which may, for example, be connected to a path which passes through humidification chamber 110. The valve is switched by the movement of valve shuttle 2 therein, which experiences a rotational as well as longitudinal movement within the valve body 1, guided by the movement of spindles 3 defined upon valve shuttle 2 within slots 6 defined on the interior surface of valve body 1, as best shown in FIGS. 4 and 5.

Coupler 5, positioned at the inlet port 10 of valve body 1 serves as an interface to a hose or other conduit carrying the air from the air pump within the machine. Rare earth magnets 4 positioned on the interior of valve shuttle 2 causes valve shuttle 2 to move longitudinally within valve body 1 and responds to a magnetic field provided by a solenoid (not shown). As valve shuttle 2 moves longitudinally through valve body 1 it is also caused to rotate approximately 45 degrees by the movement of spindles 3 through slots 6.

FIG. 4 shows the device in the first state wherein airflow entering inlet port 10 exits valve body 1 through outlet port 11. In this state, opening 8a in valve shuttle 2 is aligned with outlet port 11. FIG. 5 shows the device in the second state wherein valve shuttle 2 has moved longitudinally and rotationally such that opening 8b in valve shuttle 2 is aligned with outlet port 12, allowing airflow entering inlet port 10 exits value body 1 through outlet port 12.

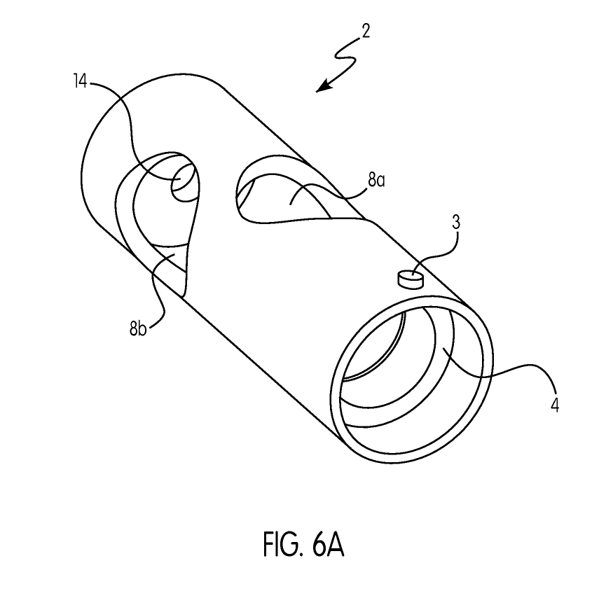

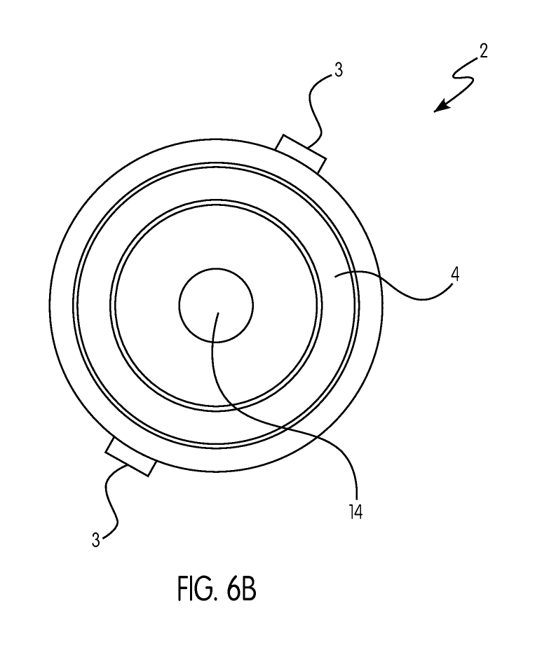

FIGS. 6a and 6b show a perspective and end view of valve shuttle 2 respectively. In FIG. 6a the relative positions of outlet holes 8a and 8b defined in the walls of valve shuttle 2 can be seen. The holes are longitudinally offset from each other and rotationally offset approximately 45 degrees from each other such that when the valve shuttle 2 moves rotationally and longitudinally within valve body 1 either hole 8a will be lined up with outlet 11 or hole 8b will be lined up with output port 12.

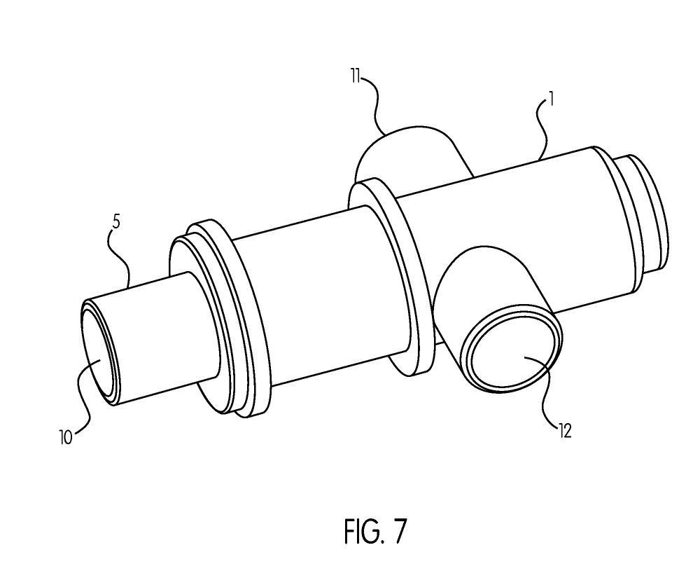

FIG. 6B shows an end view of valve shuttle 2 showing both spindles 3, rare earth magnets 4 and a hole 60 find in the distal end of valve shuttle 2 which allows movement of a valve shuttle 2 towards the closed end of valve body 1. FIG. 7 shows an outside view of the preferred embodiment of the valve.

As previously stated, there are many designs of electromechanical valves which may be used in this application and as such the invention is not meant to be limited by this particular valve. In addition, the method and apparatus of the invention is not limited to use in CPAP or Bi-PAP machines, but has applications in any machine delivering air to a patient, such as a ventilator or for use during surgery to deliver anesthesia.

We claim:

1. A breathing gas delivery machine comprising:
a. a blower;
b. a humidification chamber;
c. an airflow pathway for directing airflow from said blower to an airflow outlet; and
d. a valve, positioned in said airflow path, wherein said valve allows airflow to pass through said humidification chamber during an inhalation phase of a breathing cycle of a user of said machine and further wherein said valve diverts said airflow around said humidification chamber during an exhalation phase of a breathing cycle of a user of said machine.

2. The breathing gas delivery machine of claim 1 further comprising:
a. a microprocessor; and
b. software, execute by said microprocessor, said software performing the functions of:
controlling said blower;
switching said valve between a first state which allows said airflow to pass through said humidification chamber and a second state which divert said airflow away from said humidification chamber; and
detecting transitions between the inhalation phase and the exhalation phase of the breathing cycle of a user of said machine.

3. The breathing gas delivery machine of claim 1 further comprising one or more sensors, positioned in said airflow path, for providing data to said microprocessor, said data being used to detect the transitions in the breathing cycle.

4. The breathing gas delivery machine of claim 3 wherein said sensors are selected from a group consisting of a pressure sensor and an air flow sensor.

5. The breathing gas delivery machine of claim 2:
a. wherein said valve is switched from said first state to said second state when a transition from the inhalation phase to the exhalation phase of the breathing cycle is detected; and
b. wherein said valve is switched between said second state and said first state when a transition from the exhalation phase to the inhalation phase of the breathing cycle is detected.

6. The breathing gas delivery machine of claim 2:
a. wherein said valve is switched from said first state to said second state at a first time prior to the detection of the transition from the inhalation phase to the exhalation phase of the breathing cycle; and
b. wherein said valve is switched between said second state and said first state at a second time prior to the detection of the transition from the exhalation phase to the inhalation phase of the breathing cycle.

7. The breathing gas delivery machine of claim 6:
wherein said first time is roughly equal to the time it takes for airflow to travel between said valve and said airflow outlet during said inhalation phase; and
wherein said second time is roughly equal to the time it takes for airflow to travel between said valve and said airflow outlet during said exhalation phase.

8. The breathing gas delivery machine of claim 7 wherein said first time and said second time may be lengthened or shortened depending upon the amount of leakage being experienced by a user of said machine.

9. The breathing gas delivery machine of claim 2 wherein said value comprises:
a. a cylindrical body having an inlet, a first outlet and a second outlet defined thereon, said inlet defined at one end of said cylindrical body and said opposite end of said cylindrical body being closed, and said first and second outlets being defined on a wall of said cylindrical body; and
b. a cylindrical shuttle, moveable within said valve body, said shuttle having opening at both ends thereof, and a first hole and a second hole defined in a side wall thereof;
wherein said first state is defined by said shuttle being in a position wherein said first hole is aligned with said first outlet and wherein said second hole is not aligned with said second outlet; and
wherein said second state is defined by said shuttle being in a position wherein said second hole is aligned with said second outlet and wherein said first hole is not aligned with said first outlet.

10. The breathing gas delivery machine of claim 9 wherein said valve further comprises means for moving said shuttle back and forth within said body.

11. The breathing gas delivery machine of claim 10 wherein said means for moving is a solenoid.

12. The breathing gas delivery machine of claim 9 wherein the movement of said shuttle within said body comprises both longitudinal and rotational movement.

13. The breathing gas delivery machine of claim 9 wherein the movement of said shuttle within said body is guided by a tab defined on the outside wall of said shuttle and a groove define in the inside wall of said body such that said tab rides within said groove.

14. The breathing gas delivery machine of claim 9 wherein said groove is shaped such as to impart both longitudinal and rotational movement to said shuttle with respect to said body.

15. The breathing gas delivery machine of claim 9 wherein said inlet, said first outlet and said second outlet define shoulders therearound, to facilitate the connection of hoses comprising said airflow pathway.
